(19) United States
(12) Patent Application Publication
(10) Pub. No.: US 2021/0050114 A1
(43) Pub. Date: Feb. 18, 2021

(54) METHOD AND DEVICE FOR CONSTRUCTING DIGITAL DISEASE MODULE

(71) Applicant: Industrial Technology Research Institute, Hsinchu (TW)

(72) Inventors: Shu-Yi YIN, New Taipei City (TW); I-Hong PAN, Zhubei City (TW)

(73) Assignee: Industrial Technology Research Institute, Hsinchu (TW)

(21) Appl. No.: 16/923,930

(22) Filed: Jul. 8, 2020

Related U.S. Application Data
(60) Provisional application No. 62/887,869, filed on Aug. 16, 2019.

(30) Foreign Application Priority Data
Dec. 25, 2019 (TW) 108147515

(51) Int. Cl. G16H 50/50; G16H 50/70; G16B 40/20

(52) U.S. Cl. CPC G16H 50/50; G16H 50/20; G16B 40/20; G16H 50/70

(57) ABSTRACT

A method for constructing a digital disease module includes: determining the relationship between changes of gene/protein expression level of a gene and a disease as a first positive-negative correlation coefficient; determining the relationship between appearance of SNP and the disease as a second positive-negative correlation coefficient; determining a gene product of the gene which is a target for disease suppression as a third positive-negative correlation coefficient; determining results of text mining for functions/activities of the gene product of the gene corresponding to the disease as a fourth positive-negative correlation coefficient; determining whether the gene is the upstream gene of the signaling transduction pathway and the relation with the disease as a fifth positive-negative correlation coefficient; adding any three or more coefficients into a first sum of coefficients; and constructing a digital disease module based on the first sum of coefficients to present disease genomic information.

### CROSS REFERENCE TO RELATED APPLICATIONS

The present application claims priority from U.S. Provisional Application filed on Aug. 16, 2019 in the United States Patent and Trademark Office and assigned Ser. Nos. 62/887,869, and from Taiwan Patent Application No. 108147515, filed on Dec. 25, 2019, the entirety of which are incorporated herein by reference.

### TECHNICAL FIELD

The present disclosure is in the field of systems biology, bioinformatics, and gene/protein processing, and more particularly, it relates to a method and device for constructing a digital disease module.

### BACKGROUND

In general, the genomics-related data of specific human diseases is complex. Since genes have different patterns and units, it is difficult to integrate analysis and perform fast calculations. The clinical translation effect of genomics-related data in cell and animal experimental models is limited. In addition, the evaluation effectiveness, range, and speed of current living efficacy test systems are limited.

Therefore, there is a need for a method and a device for constructing a digital disease module in order to compile a plurality of relevant information of specific diseases/physiological phenomenon, which can then serve as a basis for rapid comparison to evaluate drug activity and clinical translatability.

### SUMMARY

The following summary is illustrative only and is not intended to be limiting in any way. That is, the following summary is provided to introduce concepts, highlights, benefits and advantages of the novel and non-obvious techniques described herein. Select, not all, implementations are described further in the detailed description below. Thus, the following summary is not intended to identify essential features of the claimed subject matter, nor is it intended for use in determining the scope of the claimed subject matter.

Therefore, the main purpose of the present disclosure is to provide a method and a device for constructing a digital disease module to improve the above disadvantages.

In an embodiment, a method for constructing a digital disease module is provided in the disclosure. The method comprises: determining the relationship between changes of gene/protein expression level of a gene and a disease as a first positive-negative correlation coefficient (Ve); determining the relationship between an appearance of single nucleotide polymorphisms (Appearance of SNP) of the gene and the disease as a second positive-negative correlation coefficient (Vm); determining a gene product of the gene which is a target for disease suppression as a third positive-negative correlation coefficient (Vt); determining results of text mining for functions/activities of the gene product of the gene corresponding to the disease as a fourth positive-negative correlation coefficient (Vr); determining whether the gene is the upstream gene of the signaling transduction pathway and the relation with the disease as a fifth positive-negative correlation coefficient (Vu); adding any three or more positive-negative correlation coefficients into a first sum of coefficients; and constructing a digital disease module based on the first sum of coefficients to present disease genomic information.

In an embodiment, a device for constructing a digital disease module is provided. The device comprises at least one processor and at least one computer storage media for storing at least one computer-readable instruction. The processor is configured to drive the computer storage media to execute the following tasks: determining the relationship between changes of gene/protein expression level of a gene and a disease as a first positive-negative correlation coefficient (Ve); determining the relationship between an appearance of single nucleotide polymorphisms (Appearance of SNP) of the gene and the disease as a second positive-negative correlation coefficient (Vm); determining a gene product of the gene which is a target for disease suppression as a third positive-negative correlation coefficient (Vt); determining results of text mining for functions/activities of the gene product of the gene corresponding to the disease as a fourth positive-negative correlation coefficient (Vr); determining whether the gene is the upstream gene of the signaling transduction pathway and the relation with the disease as a fifth positive-negative correlation coefficient (Vu); adding any three or more positive-negative correlation coefficients into a first sum of coefficients; and constructing a digital disease module based on the first sum of coefficients to present disease genomic information.

In an embodiment, a method for constructing a digital disease module is provided. The method comprises: determining the relationship between changes of gene/protein expression level of a gene and a disease as a first positive-negative correlation coefficient (Ve); determining the relationship between an appearance of single nucleotide polymorphisms (Appearance of SNP) of the gene and the disease as a second positive-negative correlation coefficient (Vm); determining a gene product of the gene which is a target for disease suppression as a third positive-negative correlation coefficient (Vt); determining results of text mining for functions/activities of the gene product of the gene corresponding to the disease as a fourth positive-negative correlation coefficient (Vr); determining whether the gene is the upstream gene of the signaling transduction pathway and the relation with the disease as a fifth positive-negative correlation coefficient (Vu); adding any two or more positive-negative correlation coefficients into a first sum of coefficients; and constructing a digital disease module based on the first sum of coefficients to present disease genomic information.

In an embodiment, a method for constructing a digital disease module is provided. The method comprises: determining the relationship between changes of gene/protein expression level of a gene and a disease as a first positive-negative correlation coefficient (Ve); determining the relationship between an appearance of single nucleotide polymorphisms (Appearance of SNP) of the gene and the disease as a second positive-negative correlation coefficient (Vm); determining a gene product of the gene which is a target for disease suppression as a third positive-negative correlation coefficient (Vt); determining results of text mining for functions/activities of the gene product of the gene corresponding to the disease as a fourth positive-negative correlation coefficient (Vr); determining whether the gene is the upstream gene of the signaling transduction pathway and the relation with the disease as a fifth positive-negative correlation coefficient (Vu); adding any one or more positive-negative correlation coefficients into a first sum of coefficients; and constructing a digital disease module based on the first sum of coefficients to present disease genomic information.

### BRIEF DESCRIPTION OF DRAWINGS

The accompanying drawings are included to provide a further understanding of the disclosure, and are incorporated in and constitute a part of the present disclosure. The drawings illustrate implementations of the disclosure and, together with the description, serve to explain the principles of the disclosure. It should be appreciated that the drawings are not necessarily to scale as some components may be shown out of proportion to the size in actual implementation in order to clearly illustrate the concept of the present disclosure.

FIG. 1 is a flowchart illustrating a method for constructing a digital disease module according to an embodiment of the disclosure.

FIG. 2 is a schematic diagram illustrating the conversion of the digital disease module matrix to the digital disease module according to an embodiment of the present disclosure.

FIG. 3 is a schematic diagram illustrating a digital insomnia disease module 300 constructed by adding only the first, second and fifth positive-negative correlation coefficients as a sum of coefficients according to an embodiment of the present disclosure.

FIG. 4 is a schematic diagram illustrating a digital insomnia disease module 400 constructed by adding only the first, second, third, and fifth positive and negative correlation coefficients according to an embodiment of the present disclosure.

FIG. 5 illustrates an exemplary operating environment for implementing embodiments of the present disclosure.

### DETAILED DESCRIPTION

Various aspects of the disclosure are described more fully below with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

The word “exemplary” is used herein to mean “serving as an example, instance, or illustration.” Any aspect described herein as “exemplary” is not necessarily to be construed as preferred or advantageous over other aspects. Furthermore, like numerals refer to like elements throughout the several views, and the articles “a” and “the” includes plural references, unless otherwise specified in the description.

It should be understood that when an element is referred to as being “connected” or “coupled” to another element, it may be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion. (e.g., “between” versus “directly between”, “adjacent” versus “directly adjacent”, etc.).

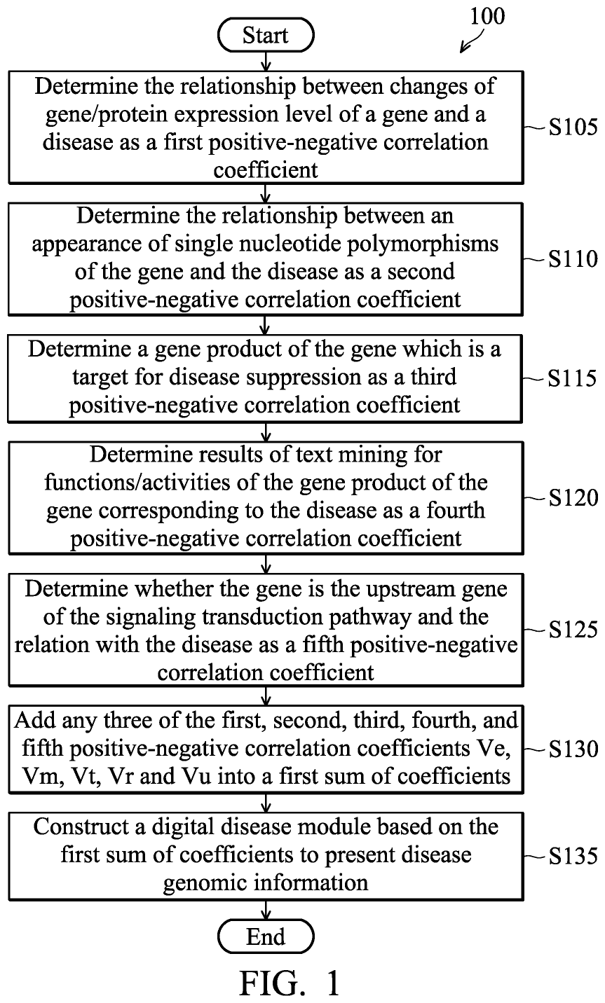

FIG. 1 is a flowchart illustrating a method 100 for constructing a digital disease module according to an embodiment of the disclosure. The method can be executed by an electronic device. The types of electronic devices range from small handheld devices (e.g, mobile phones to portable computers) to mainframe systems (e.g, mainframe computers) or central processing units. Examples of portable computers include devices such as personal digital assistants (PDAs), notebook computers, and the like.

In step S105, the electronic device determines the relationship between changes of gene/protein expression level of a gene and a disease as a first positive-negative correlation coefficient (Ve). Specifically, it is assumed that the number of patients with a disease caused by a gene/protein product is x, and the number of normal persons without the disease caused by the gene/protein product is y. The electronic device determines the first positive-negative correlation coefficient (Ve) of a gene corresponding to the gene/protein product based on a first expression level statistical value a1, a2, a3, . . . , ax (hereinafter abbreviated as an) of the gene/protein product of the gene when the disease occurs and a second expression level statistical value b1, b2, b3, . . . , by (hereinafter abbreviated as bn) of the gene/protein product when the disease does not occur, wherein the first expression level statistical values a1 a2, a3, . . . , ax respectively represent the gene/protein expression levels of patients from 1 to x, and the second expression level statistical values b1, b2, b3, . . . , by respectively represent the gene/protein expression levels of normal people from 1 to y. Specifically, the first expression level statistical value (a1, a2, a3, . . . , ax) and the second expression level statistical value (b1, b2, b3, . . . , by) are values of specific gene/protein expression levels (Codename).

When the first average, Average (a1: ax), of the first expression level statistical value, an, divided by the second average, Average (b1: by), of the second expression level statistical value, bn, is greater than or equal to 2 and the first statistical difference between the first expression level statistical value an and the second expression level statistical value bn is significant (equivalent to an independent two-sample T test (a1: ax, b1: by) less than 0.05), the electronic device gives or determines the first positive-negative correlation coefficient Ve of the gene as Ve1 (i.e., Ve=Ve1), wherein Ve1 is a positive correlation score greater than 0, for example, Ve1=2. That is, both conditions “Average (a1: ax)/ Average (b1: by)≥2” and “T test (a1: ax, b1: by)<0.05” are met.

When the first average, Average (a1: ax), of the first expression level statistical value, an, divided by the second average, Average (b1: by), of the second expression level statistical value, bn, is less than 2 and greater than 1 (the mathematical formula: 2>Average (a1: ax)/Average (b1: by)>1) and the first statistical difference is significant (equivalent to the independent two-sample T test (a1: ax, b1: by) less than 0.05), the electronic device gives or determines the first positive-negative correlation coefficient Ve of the gene as Ve2 (i.e., Ve=Ve2), wherein Ve2 is a positive correlation score greater than 0, for example, Ve2=1. That is, both conditions “2>Average (a1: ax)/Average (b1: by)>1” and “T test (a1: ax, b1: by)<0.05” are met.

When the first statistical difference is not significant (that is, the correlation between the gene/protein expression level trend and the appearance of disease is not significant), the independent two-sample T test value is greater than or equal to 0.05 (the mathematical formula: T test (a1: ax, b1: by) 0.05) and the electronic device gives or determines the first positive-negative correlation coefficient Ve of the gene as 0 (i.e., Ve=0).

When the first average, Average (a1: ax), of the first expression level statistical value, an, divided by the second average, Average (b1: by), of the second expression level statistical value, bn, is less than 1 and greater than 0.5 (the mathematical formula: 1>Average (a1: ax)/Average (b1: by)>0.5) and the first statistical difference is significant (equivalent to the independent two-sample T test (a1: ax, b1: by) less than 0.05), the electronic device gives or determines the first positive-negative correlation coefficient Ve of the gene as Ve3 (i.e., Ve=Ve3), wherein Ve3 is a negative correlation score less than 0, for example, Ve3=−1. That is, both conditions “1>Average (a1: ax)/Average (b1: by)>0.5” and “T test (a1: ax, b1: by)<0.05” are met.

When the first average, Average (a1: ax), of the first expression level statistical value, an, divided by the second average, Average (b1: by), of the second expression level statistical value, bn, is less than or equal to 0.5 (the mathematical formula: 0.5 Average (a1: ax)/Average (b1: by)) and the first statistical difference is significant (equivalent to the independent two-sample T test (a1: ax, b1: by) less than 0.05), the electronic device gives or determines the first positive-negative correlation coefficient Ve of the gene as Ve4 (i.e., Ve=Ve4), wherein Ve4 is a negative correlation score less than 0, for example, Ve4=−2. That is, both conditions “0.5≥Average (a1: ax)/Average (b1: by)” and “T test (a1: ax, b1: by)<0.05” are met.

In this embodiment, the first positive-negative correlation coefficient (Ve) of a gene corresponding to the gene/protein product has a numerical relationship: Ve1>Ve2>0>Ve3>Ve4. It should be noted that the values of Ve1, Ve2, Ve3, and Ve4 are not intended to limit the present disclosure, and those skilled in the art can make appropriate replacements or adjustments according to this embodiment.

How to determine whether the first statistical difference between the first expression level statistical value an and the second expression level statistical value bn is significant will be explained as follows. Since the number of samples of the first expression level statistical value an and the number of samples of the second expression level statistical value bn are different, the first statistical difference can be calculated using an independent two-sample (T test) formula. When two groups of independent samples, the first expression level statistical value an and the second expression level statistical value bn, have the same or different sample numbers x and y, respectively, and the first expression level statistical value an and the second expression level statistical value bn are independent from each other and from two normal distributions with unequal variation numbers, the formula (1) of independent two-sample is as follows:

$$t = \frac{\overline{an} - \overline{bn} - {μ}_{0}}{\sqrt{\frac{{s}_{1}^{2}}{x} + \frac{{s}_{2}^{2}}{y}}} \qquad ( 1 )$$

wherein the averages of the two groups of samples: a̅n̅=(Σᵢ₌₁^{x}aᵢ)/x, b̅n̅=(Σⱼ₌₁^{y}bⱼ)/y, the variation numbers of two groups of samples: s₁²=(Σᵢ₌₁^{x}(aᵢ−a̅n̅)²)/(x−1) and s₂²=(Σⱼ₌₁^{y}(bⱼ=b̅n̅)/(y=1). When the independent two-sample T test (a1: ax, b1: by) is less than 0.05, the electronic device determines that the first statistical difference between the first expression level statistical value an and the second expression level statistical value bn is significant. When the independent two-sample T test (a1: ax, b1: by) is greater than or equal to 0.05, the electronic device determines that the first statistical difference between the first expression level statistical value an and the second expression level statistical value bn is not significant.

Then, in step S110, the electronic device determines the relationship between an appearance of single nucleotide polymorphisms (Appearance of SNP) of the gene and the disease as a second positive-negative correlation coefficient (Vm). Specifically, the electronic device determines statistical values of appearance rate of SNP of a gene sequence of the gene as c1, c2, c3, . . . , cx, and determines statistical values of appearance rate of no SNP as d1, d2, b3, . . . , dy. In one embodiment, the appearance rate of SNP can be expressed as a percentage or a fraction.

When the gene has more than two appearances of SNP that are negatively correlated with an appearance/occurrence of the disease (that is, when this gene mutation promotes or causes the disease/physiological phenomenon, it is determined as a negative correlation), a third average, Average (c1: cx), of the statistical values of appearance rate of SNP divided by a fourth average, Average (d1: dy), of the statistical value of appearance rate of no SNP is greater than 1 (the mathematical formula: Average (c1: cx)/Average (d1: dy)>1), and the second statistical difference between the statistical values of appearance rate of SNP and the statistical values of appearance rate of no SNP is significant (the mathematical formula: an independent two-sample T test (c1: cx, d1: dy) of SNP<0.05), the electronic device determines the second positive-negative correlation coefficient Vm of the gene as Vm1 (i.e., Vm=Vm1), wherein Vm1 is a positive correlation score greater than 0, for example, Vm1=2. That is, conditions “more than two appearances of SNP that are negatively correlated with an appearance/occurrence of the disease”, “Average (c1: cx)/ Average (d1: dy)>1” and “T test (c1: cx, d1: dy)<0.05” are met.

When the gene has more than one appearance of SNP that is negatively correlated with an appearance of the disease, a third average, Average (c1: cx), of the statistical values of appearance rate of SNP divided by a fourth average, Average (d1: dy), of the statistical values of appearance rate of no SNP is greater than 1 (the mathematical formula: Average (c1: cx)/Average (d1: dy)>1), and the second statistical difference is significant (the mathematical formula: the independent two-sample T test (c1: cx, d1: dy) of SNP<0.05), the electronic device gives or determines the second positive-negative correlation coefficient Vm of the gene as Vm2 (i.e., Vm=Vm2), wherein Vm2 is a positive correlation score greater than 0, for example, Vm2=1. That is, conditions “more than one appearance of SNP that is negatively correlated with an appearance/occurrence of the disease”, “Average (c1: cx)/Average (d1: dy)>1” and “T test (c1: cx, d1: dy)<0.05” are met.

When any appearances of SNP of the gene are not correlated with an appearance/occurrence of the disease, and also the second statistical difference is not significant, the independent two-samples T test value is greater than or equal to 0.05 (the mathematical formula: T test (c1: cx, d1: dy)>0.05), the electronic device gives or determines the second positive-negative correlation coefficient Vm of the gene as 0 (i.e., Vm=0).

When the gene has more than one appearance of SNP that is positively correlated with the appearance of the disease (that is, when this gene mutation reduces or inhibits the target disease/physiological phenomenon, it is determined as a positive correlation), a third average, Average (c1: cx), of the statistical values of appearance rate of SNP divided by a fourth average, Average (d1: dy), of the statistical values of appearance rate of no SNP is greater than 1 (the mathematical formula: Average (c1: cx)/Average (d1: dy)>1), and the second statistical difference is significant (the mathematical formula: an independent two-sample T test (c1: cx, d1: dy) of SNP<0.05), the electronic device gives or determines the second positive-negative correlation coefficient Vm of the gene as Vm3 (i.e., Vm=Vm3), wherein Vm3 is a negative correlation score less than 0, for example, Vm3=−1. That is, conditions “more than one appearance of SNP that is positively correlated with an appearance/occurrence of the disease”, “Average (c1: cx)/Average (d1: dy)>1” and “T test (c1: cx, d1: dy)<0.05” are met.

When the gene has more than two appearances of SNP that are positively correlated with the appearance of the disease, a third average, Average (c1: cx), of the statistical values of appearance rate of SNP divided by a fourth average, Average (d1: dy), of the statistical values of appearance rate of no SNP is greater than 1 (the mathematical formula: Average (c1: cx)/Average (d1: dy)>1), and the second statistical difference is significant (the mathematical formula: an independent two-sample T test (c1: cx, d1: dy) of SNP<0.05), the electronic device gives or determines the second positive-negative correlation coefficient Vm of the gene as Vm4 (i.e., Vm=Vm4), wherein Vm4 is a negative correlation score less than 0, for example, Vm4=−2. That is, conditions “more than one appearance of SNP that is positively correlated with an appearance/occurrence of the disease”, “Average (c1: cx)/Average (d1: dy)>1” and “T test (c1: cx, d1: dy)<0.05” are met.

In the embodiment, the second positive-negative correlation coefficient Vm of the appearance of SNP of the gene corresponding to the disease has a numerical relationship: Vm1>Vm2>0>Vm3>Vm4. It should be noted that the values of Vm1, Vm2, Vm3, and Vm4 are not intended to limit the present disclosure, and those skilled in the art can make appropriate replacements or adjustments according to this embodiment. Regarding how to determine whether the second statistical difference between the statistical value of the appearance of SNP and the statistical value of appearance rate of no SNP is significant, please refer to the description of formula (1) and the details related to the formula (1) will be omitted.

In step S115, the electronic device gives or determines a gene product of a gene which is a target for disease suppression as a third positive-negative correlation coefficient (Vt). Specifically, the electronic device determines whether a gene product (derived from a gene) is a therapeutic target of a known antagonist or a therapeutic target of a known agonist to determine the third positive-negative correlation coefficient (Vt) of a gene corresponding to the gene product.

When the gene product of the gene is a therapeutic target of a known antagonist, and the known antagonist is a drug for a known disease, the electronic device determines the third positive-negative correlation coefficient Vt of the gene as Vt1 (i.e., Vt=Vt1), wherein Vt1 is a positive correlation score greater than 0, for example, Vt1=3.

When the gene product is a therapeutic target of a known antagonist, and the known antagonist is a clinical trial drug (that is, a candidate for clinical trials from Phase I to Phase III), the electronic device determines the third positive-negative correlation coefficient Vt of the gene as Vt2 (i.e., Vt=Vt2), wherein Vt2 is a positive correlation score greater than 0, for example, Vt2=2.

When the gene product is a therapeutic target of a known antagonist, and the known antagonist is not a clinical trial drug, the electronic device determines the third positive-negative correlation coefficient Vt of the gene as Vt3 (i.e., Vt=Vt3), wherein Vt3 is a positive correlation score greater than 0, for example, Vt3=1.

When the gene product is not an antagonist of a specific disease or the gene product is not a therapeutic target of an agonist, the electronic device determines the third positive-negative correlation coefficient Vt of the gene as 0 (i.e., Vt=0).

When the gene product is a therapeutic target of a known agonist, and the known agonist is not a clinical trial drug, the electronic device determines the third positive-negative correlation coefficient Vt of the gene as Vt4 (i.e., Vt=Vt4), wherein Vt4 is a negative correlation score less than 0, for example, Vt4=−1.

When the gene product is a therapeutic target of a known agonist, and the known agonist is a clinical trial drug (that is, a candidate for clinical trials from Phase Ito Phase III), the electronic device determines the third positive-negative correlation coefficient Vt of the gene as Vt5 (i.e., Vt=Vt5), wherein Vt5 is a negative correlation score less than 0, for example, Vt5=−2.

When the gene product is a therapeutic target of a known agonist, and the known agonist is a drug for a known disease, the electronic device determines the third positive-negative correlation coefficient Vt of the gene as Vt6 (i.e., Vt=Vt6), wherein Vt6 is a negative correlation score less than 0, for example, Vt6=−3.

In the embodiment, the third positive-negative correlation coefficient Vt of a gene product that is a target for inhibiting the disease has the following relationship: Vt1>Vt2>Vt3>0>Vt4>Vt5>Vt6. It should be noted that the values of Vt1, Vt2, Vt3, Vt4, Vt5 and Vt6 are not intended to limit the present disclosure, and those skilled in the art can make appropriate replacements or adjustments according to this embodiment.

Next, in step S120, the electronic device determines results of text mining for functions/activities of the gene product of the gene corresponding to the disease as a fourth positive-negative correlation coefficient (Vr). Specifically, the electronic device compiles textual or narrative data about the functions/activities of the gene and the appearance/occurrence of the disease through a document exploration technology, and determines the fourth positive-negative correlation coefficient (Vr) corresponding a gene of the functions/activities.

For the functions/activities of the gene product of a gene, when there is literature (thesis or journal) description, inference or experiment confirming that the functions/activities of the gene product are positively correlated with the appearance of the disease or is not beneficial to the treatment of the disease (specifically, when there is literature describing in text that a gene or a gene product is positively related to the appearance of the disease, or that a gene or gene product is not beneficial to the treatment of the disease, the literature can be annotated using existing text exploration or manual methods), the electronic devices determines that the functions/activities of the gene product of a gene is positively correlated with the appearance of the disease, and determines the fourth positive-negative correlation coefficient Vr of the gene as Vr1 (that is, Vr=Vr1), wherein Vr1 is a positive correlation score greater than 0, For example, Vr1=2.

For the functions/activities of the gene product of a gene, when there is no literature description, inference or experiment confirming that the functions/activities of the gene product are related to the appearance of the disease, or the description of the positive-negative correlation is ambiguous, the electronic device determines the fourth positive-negative correlation coefficient Vr of the gene as 0 (that is, Vr=0).

For the functions/activities of the gene product of a gene, when there is literature description, inference or experiment confirming that the functions/activities of the gene product are negatively correlated with the appearance of the disease, or the functions/activities of the gene product are beneficial to the treatment of the disease, the electronic device determines the fourth positive-negative correlation coefficient Vr of the gene as Vr2 (i.e., Vr=Vr2), wherein Vr2 is a negative correlation score less than 0, for example, Vr2=−2.

In the embodiment, the fourth positive-negative correlation coefficient Vr of the literature survey results of the functions/activities of the gene product corresponding to the disease has the following relationship: Vr1>0>Vr2. It should be noted that the values of Vr1 and Vr2 are not intended to limit the present disclosure, and those skilled in the art can make appropriate replacements or adjustments according to this embodiment.

In step S125, the electronic device determines whether the gene is the upstream gene of the signaling transduction pathway and the relation with the disease as a fifth positive-negative correlation coefficient (Vu). The details are described as follows.

When the gene product of a gene is an extracellular ligand, a cell surface receptor or a transcription factor, the electronic device determines that the gene belongs to and is classified as the upstream gene. Furthermore, the electronic device adds the first, second, third, and fourth positive-negative correlation coefficients of the gene Ve, Vm, Vt, and Vr obtained in steps S105, S110, S115, and S120 into a second sum of coefficients.

When the second sum of coefficients of the first, second, third and fourth positive-negative correlation coefficients of the gene Ve, Vm, Vt and Vr is positive (that is, Ve+Vm+Vt+Vr>0) and the gene belongs to the upstream gene (the gene product is an extracellular ligand, a cell surface receptor, or a transcription factor), the electronic device gives or determines the fifth positive-negative correlation coefficient Vu of the gene as Vu1 (that is, Vu=Vu1), wherein Vu1 is a positive correlation score greater than 0, for example, Vu1=1.

When the second sum of coefficients of the first, second, third and fourth positive-negative correlation coefficients of the gene Ve, Vm, Vt and Vr is 0 (the mathematical formula: Ve+Vm+Vt+Vr=0) or the gene does not belong to the upstream gene (that is, the gene product is not an extracellular ligand, a cell surface receptor, or a transcription factor), the electronic device gives or determines the fifth positive-negative correlation coefficient Vu of the gene as 0 (that is, Vu=0).

When the second sum of coefficients of the first, second, third and fourth positive-negative correlation coefficients of the gene Ve, Vm, Vt and Vr is negative (that is, Ve+Vm+Vt+Vr<0) and the gene belongs to the upstream gene (the gene product is an extracellular ligand, a cell surface receptor, or a transcription factor), the electronic device gives or determines the fifth positive-negative correlation coefficient Vu of the gene as Vu2 (that is, Vu=Vu2), wherein Vu2 is a negative correlation score less than 0, for example, Vu2=−1.

In the embodiment, the fifth positive-negative correlation coefficient Vu of the gene that is the upstream gene of the signaling transduction pathway corresponding to the disease has the following relationship: Vu1>0>Vu2. It should be noted that the values of Vu1 and Vu2 are not intended to limit the present disclosure, and those skilled in the art can make appropriate replacements or adjustments according to this embodiment.

In step S130, the electronic device adds any three of the first, second, third, fourth, and fifth positive-negative correlation coefficients Ve, Vm, Vt, Vr and Vu into a first sum of coefficients. In another embodiment, the electronic device may also add any four of the first, second, third, fourth, and fifth positive-negative correlation coefficients Ve, Vm, Vt, Vr and Vu or add the first, second, third, fourth, and fifth positive-negative correlation coefficients Ve, Vm, Vt, Vr and Vu into a first sum of coefficients G (that is, G=Ve+Vm+Vt+Vr+Vu).

Finally, in step S135, the electronic device constructs a digital disease module according to the first sum of coefficients G to present disease genomic information, wherein the digital disease module is a three-dimensional model.

In an embodiment, the positive-negative correlation coefficients meet the following conditions, and the maximum value of each positive and negative correlation score has the following inequality relationship:

(Vm1+Vt1+Vr1)>Ve1;

(Ve1+Vt1+Vr1)>Vm1;

(Ve1+Vm1+Vr1)>Vt1;

(Ve1+Vm1+Vt1)>Vr1; and

(Ve1+Vm1+Vt1+Vr1)>Vu1.

The sum of the maximum values of any three or four positive and negative correlation scores is greater than the maximum value of the other single positive and negative correlation score.

In an embodiment, the minimum value of each positive and negative correlation score has the following inequality relationship:

Ve4>(Vm4+Vt6+Vr2);

Vm4>(Ve4+Vt6+Vr2);

Vt6>(Ve4+Vm4+Vr2);

i Vr2>(Ve4+Vm4+Vt6); and

Vu2>(Ve4+Vm4+Vt6+Vr2).

The sum of the minimum values of any positive and negative correlation scores is greater than the maximum value of a single positive and negative correlation score. The minimum value of any positive or negative correlation score is greater than the sum of the minimum values of the other three or four positive and negative correlation scores.

It should be understood that the significance of the inequality is that any one of the first, second, third, fourth, and fifth positive-negative correlation coefficients Ve, Vm, Vt, Vr and Vu does not dominate the total coefficient G.

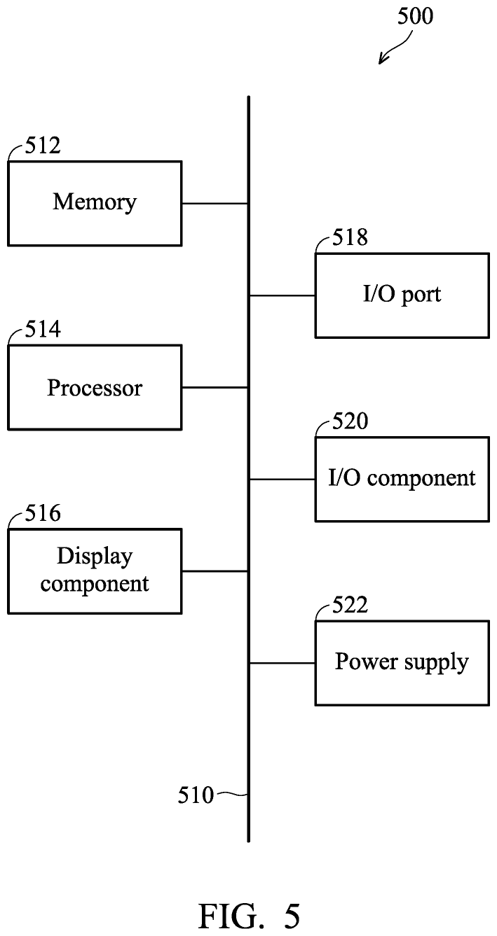

Each component of the electronic device that executes the method of constructing the digital disease module in FIG. 1 can be implemented via any type of computing device, such as a computer or a microprocessor, such as the computing device 500 described with reference to FIG. 5, for example.

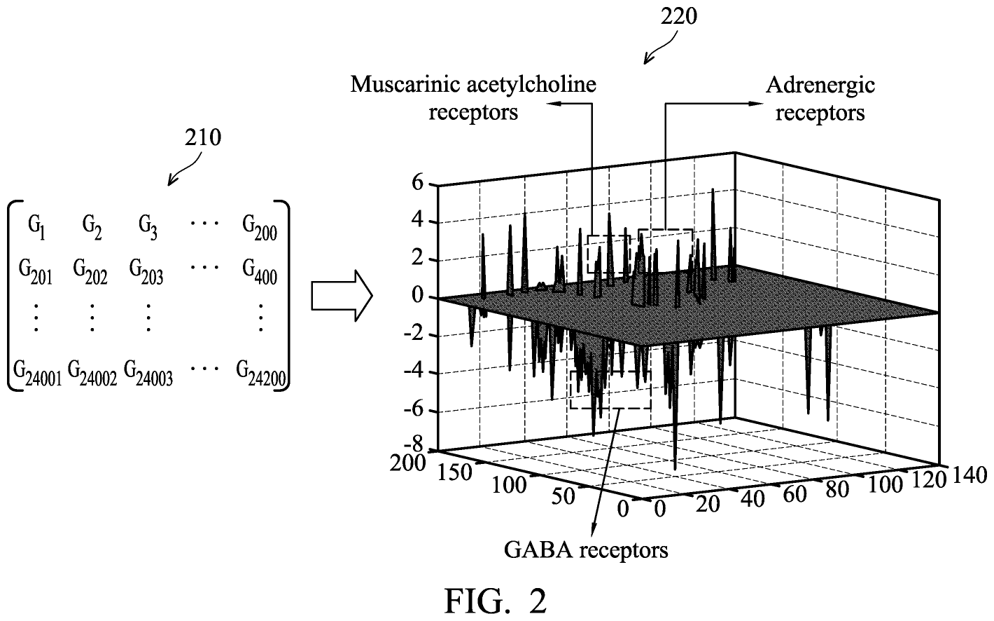

FIG. 2 is a schematic diagram illustrating the conversion of the digital disease module matrix 210 to the digital disease module 220 according to an embodiment of the present disclosure. Human insomnia is used as an example in the embodiment. It is assumed that in the embodiment, there is a sum of coefficients of 24200 genes. As shown in FIG. 2, the digital disease module matrix 210 is composed of coefficient sums G1, G2, . . . , G24000, G24001, . . . , G24200, and the sum of each coefficient Gn represents that, for a specific disease, the sum of the scores of the first, second, third, fourth, and fifth positive-negative correlation coefficients Ve, Vm, Vt, Vr and Vu of each gene or gene product. The digital disease module matrix 210 can be converted into a three-dimensional model by computer software, such as the digital disease module 220 shown in FIG. 2. As shown in FIG. 2, the peaks that are upward (such as Adrenergic receptors) have a positive correlation, and the peaks that are downward (such as GABA receptors) have a negative correlation. The multiple style information of functions/activities of gene products derived from over 24,000 human genes in specific diseases or physiological phenomena may be unified and calculated by the digital disease module 220, so as to provide the basis for rapid comparison for pathological research and drug development.

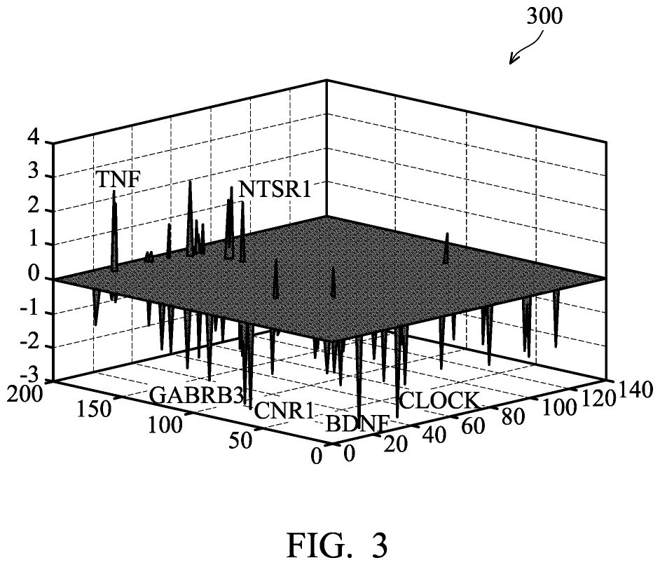

FIG. 3 is a schematic diagram illustrating a digital insomnia disease module 300 constructed by adding only the first, second and fifth positive-negative correlation coefficients Ve, Vm and Vu as a sum of coefficients according to an embodiment of the present disclosure. As shown in FIG. 3, the peaks that are upward (for example, neurotensin receptor 1 (NTSR1), tumor necrosis factor (TNF)) have a positive correlation, and the peaks that are downward (for example, GABRB3, CNR1, BDNF, CLOCK) have a negative correlation. In some embodiments, the electronic device may add any three of the first, second, third, fourth, and fifth positive-negative correlation coefficients Ve, Vm, Vt, Vr and Vu to obtain other digital insomnia disease modules 300. Therefore, the present disclosure is not limited to the schematic diagram shown in FIG. 3.

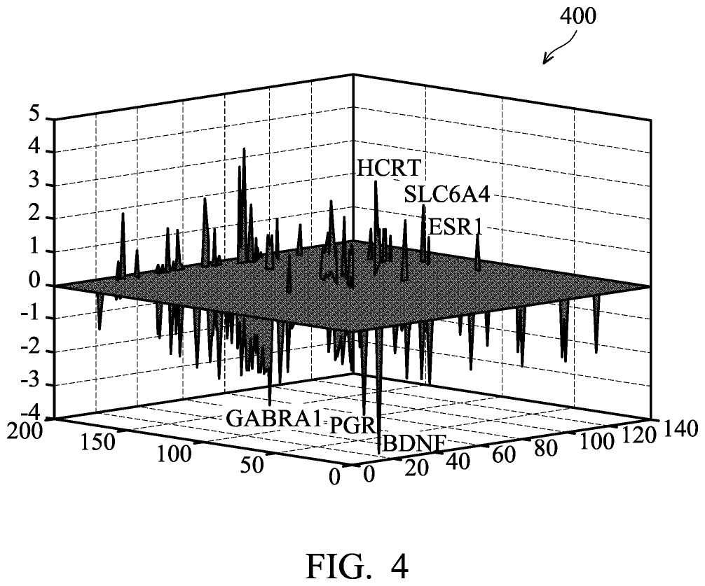

FIG. 4 is a schematic diagram illustrating a digital insomnia disease module 400 constructed by adding only the first, second, third, and fifth positive and negative correlation coefficients Ve, Vm, Vt, and Vu according to an embodiment of the present disclosure. As shown in FIG. 4, the peaks that are upward (for example, hypocretin (HCRT), SLC6A4, ESR1) have a positive correlation, and the peaks that are downward (such as GABRA1, progesterone receptor (PGR), BDNF) have a negative correlation. In some embodiments, the electronic device may add any four of the first, second, third, fourth, and fifth positive-negative correlation coefficients Ve, Vm, Vt, Vr and Vu to obtain other digital insomnia disease modules 400. Therefore, the present disclosure is not limited to the schematic diagram shown in FIG. 4.

As described above, the method and device for constructing a digital disease module provided in the disclosure may unify multi-style data including changes of gene/protein expression level of a gene/a gene product, activities/functions of a gene product, appearance of SNP of a gene, known and developing targets of disease treatment, results of literature survey, upstream genes and other data related to diseases and physiological phenomena, apply different types of genomic related data and the positive and negative correlations of specific diseases or physiological phenomena, and unify and calculate multiple-style information of more than 24,000 human gene products in specific diseases or physiological phenomena to provide a basis for rapid comparison for pathological research and drug development. In short, for a single disease, the relationships between all genes and five coefficients (gene product expression level, data of single nucleotide polymorphisms, treatment target, indicators of literature survey, upstream genes) are summarized and scored to obtain the trend of positive and negative correlations. Through early evaluating application activities of potential materials, potential therapeutic targets of specific diseases are developed to select potential compounds/molecules and evaluate clinical relevance of experimental models.

Having described embodiments of the present disclosure, an exemplary operating environment in which embodiments of the present disclosure may be implemented is described below. Referring to FIG. 5, an exemplary operating environment for implementing embodiments of the present disclosure is shown and generally known as a computing device 500. The computing device 500 is merely an example of a suitable computing environment and is not intended to limit the scope of use or functionality of the disclosure. Neither should the computing device 500 be interpreted as having any dependency or requirement relating to any one or combination of components illustrated.

The disclosure may be realized by means of the computer code or machine-useable instructions, including computer-executable instructions such as program modules, being executed by a computer or other machine, such as a personal data assistant (PDA) or other handheld device. Generally, program modules may include routines, programs, objects, components, data structures, etc., and refer to code that performs particular tasks or implements particular abstract data types. The disclosure may be implemented in a variety of system configurations, including hand-held devices, consumer electronics, general-purpose computers, more specialty computing devices, etc. The disclosure may also be implemented in distributed computing environments where tasks are performed by remote-processing devices that are linked by a communication network.

With reference to FIG. 5, the computing device 500 may include a bus 510 that is directly or indirectly coupled to the following devices: one or more memories 512, one or more processors 514, one or more display components 516, one or more input/output (I/O) ports 518, one or more input/output components 520, and an illustrative power supply 522. The bus 510 may represent one or more kinds of busses (such as an address bus, data bus, or any combination thereof). Although the various blocks of FIG. 5 are shown with lines for the sake of clarity, and in reality, the boundaries of the various components are not specific. For example, the display component such as a display device may be considered an I/O component and the processor may include a memory.

The computing device 500 typically includes a variety of computer-readable media. The computer-readable media can be any available media that can be accessed by computing device 500 and includes both volatile and nonvolatile media, removable and non-removable media. By way of example, but not limitation, computer-readable media may comprise computer storage media and communication media. The computer storage media may include volatile and nonvolatile, removable and non-removable media implemented in any method or technology for storage of information such as computer-readable instructions, data structures, program modules or other data. The computer storage media may include, but not limit to, random access memory (RAM), read-only memory (ROM), electrically-erasable programmable read-only memory (EEPROM), flash memory or other memory technology, compact disc read-only memory (CD-ROM), digital versatile disks (DVD) or other optical disk storage, magnetic cassettes, magnetic tape, magnetic disk storage or other magnetic storage devices, or any other medium which can be used to store the desired information and which can be accessed by the computing device 500. The computer storage media may not comprise signal per se.

The communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, but not limitation, communication media includes wired media such as a wired network or direct-wired connection, and wireless media such as acoustic, RF, infrared and other wireless media or any combination thereof.

The memory 512 may include computer-storage media in the form of volatile and/or nonvolatile memory. The memory may be removable, non-removable, or a combination thereof. Exemplary hardware devices include solid-state memory, hard drives, optical-disc drives, etc. The computing device 500 includes one or more processors that read data from various entities such as the memory 512 or the I/O components 520. The display component(s) 516 present data indications to a user or other device. Exemplary presentation components include a display device, speaker, printing component, vibrating component, etc.

The I/O ports 518 allow the computing device 500 to be logically coupled to other devices including the I/O components 520, some of which may be embedded. Illustrative components include a microphone, joystick, game pad, satellite dish, scanner, printer, wireless device, etc. The I/O components 520 may provide a natural user interface (NUI) that processes gestures, voice, or other physiological inputs generated by a user. For example, inputs may be transmitted to an appropriate network element for further processing. A NUI may be implemented to realize speech recognition, touch and stylus recognition, face recognition, biometric recognition, gesture recognition both on screen and adjacent to the screen, air gestures, head and eye tracking, touch recognition associated with displays on the computing device 500, or any combination of. The computing device 500 may be equipped with depth cameras, such as stereoscopic camera systems, infrared camera systems, RGB camera systems, any combination of thereof to realize gesture detection and recognition. Furthermore, the computing device 500 may be equipped with accelerometers or gyroscopes that enable detection of motion. The output of the accelerometers or gyroscopes may be provided to the display of the computing device 500 to carry out immersive augmented reality or virtual reality.

Furthermore, the processor 514 in the computing device 500 can execute the program code in the memory 512 to perform the above-described actions and steps or other descriptions herein.

It should be understood that any specific order or hierarchy of steps in any disclosed process is an example of a sample approach. Based upon design preferences, it should be understood that the specific order or hierarchy of steps in the processes may be rearranged while remaining within the scope of the present disclosure. The accompanying method claims present elements of the various steps in a sample order, and are not meant to be limited to the specific order or hierarchy presented.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

While the disclosure has been described by way of example and in terms of the preferred embodiments, it should be understood that the disclosure is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for constructing a digital disease module, comprising:
determining the relationship between changes of gene/protein expression level of a gene and a disease as a first positive-negative correlation coefficient (Ve);
determining the relationship between an appearance of single nucleotide polymorphisms (Appearance of SNP) of the gene and the disease as a second positive-negative correlation coefficient (Vm);
determining a gene product of the gene which is a target for disease suppression as a third positive-negative correlation coefficient (Vt);
determining results of text mining for functions/activities of the gene product of the gene corresponding to the disease as a fourth positive-negative correlation coefficient (Vr);
determining whether the gene is the upstream gene of the signaling transduction pathway and the relation with the disease as a fifth positive-negative correlation coefficient (Vu);
adding any three or more positive-negative correlation coefficients into a first sum of coefficients; and
constructing a digital disease module based on the first sum of coefficients to present disease genomic information.

2. The method for constructing a digital disease module as claimed in claim 1, wherein the step of determining the relationship between the changes of gene/protein expression level of the gene and the disease as the first positive-negative correlation coefficient (Ve) further comprises:
determining a first expression level statistical value of a gene/protein product of the gene when the disease occurs;
determining a second expression level statistical value of the gene/protein product when the disease does not occur;
wherein when a first average of the first expression level statistical value divided by a second average of the second expression level statistical value is greater than or equal to 2, and a first statistical difference between the first expression level statistical value and the second expression level statistical value is significant, the first positive-negative correlation coefficient Ve of the gene is determined as Ve1, wherein the first statistical difference that is significant means that an independent two-sample T test <0.05 of the first expression level statistical value and the second expression level statistical value;
when the first average of the first expression level statistical value divided by the second average of the second expression level statistical value is less than 2 and greater than 1, and the first statistical difference is significant, the first positive-negative correlation coefficient Ve of the gene is determined as Ve2;
when the first statistical difference is not significant, the first positive-negative correlation coefficient Ve of the gene is determined as 0, wherein the first statistical difference that is not significant means that the independent two-sample T test ≥0.05;
when the first average of the first expression level statistical value divided by the second average of the second expression level statistical value is less than 1 and greater than 0.5, and the first statistical difference is significant, the first positive-negative correlation coefficient Ve of the gene is determined as Ve3; and
when the first average of the first expression level statistical value divided by the second average of the second expression level statistical value is less than or equal to 0.5, and the first statistical difference is significant, the first positive-negative correlation coefficient Ve of the gene is determined as Ve4;
wherein the coefficient relationship is Ve1>Ve2>0>Ve3>Ve4.

3. The method for constructing a digital disease module as claimed in claim 1, wherein the step of determining the relationship between the appearance of SNP of the gene and the disease as the second positive-negative correlation coefficient (Vm) further comprises:
determining statistical values of appearance rate of SNP of a gene sequence of as c1˜cx;
determining statistical values of appearance rate of no SNP as d1˜dy;
wherein when the gene has more than two appearances of SNP that are negatively correlated with an appearance of the disease, a third average of the statistical values of appearance rate of SNP divided by a fourth average of the statistical values of appearance rate of no SNP is greater than 1, and a second statistical difference between the statistical values of appearance rate of SNP and the statistical values of appearance rate of no SNP is significant, the second positive-negative correlation coefficient Vm of the gene is determined as Vm1, wherein the second statistical difference that is significant means an independent two-sample T test (c1: cx, d1: dy)<0.05 of the statistical values of appearance rate of SNP and the statistical value of appearance rate of no SNP;
when the gene has more than one appearance of SNP that is negatively correlated with the appearance of the disease, the third average divided by the fourth average is greater than 1, and the second statistical difference is significant, the second positive-negative correlation coefficient Vm of the gene is determined as Vm2;
when the second statistical difference is not significant, the second positive-negative correlation coefficient Vm of the gene is determined as 0, wherein the second statistical difference that is not significant means that the independent two-sample T test ≥0.05;
when the gene has more than one appearance of SNP that is positively correlated with the appearance of the disease, the third average divided by the fourth average is greater than 1, and the second statistical difference is significant, the second positive-negative correlation coefficient Vm of the gene is determined as Vm3;
when the gene has more than two appearances of SNP that are positively correlated with the appearance of the disease, the third average divided by the fourth average is greater than 1, and the second statistical difference is significant, the second positive-negative correlation coefficient Vm of the gene is determined as Vm4;
wherein the coefficient relationship is Vm1>Vm2>0>Vm3>Vm4.

4. The method for constructing a digital disease module as claimed in claim 1, wherein the step of determining the gene product of the gene which is the target for disease suppression as the third positive-negative correlation coefficient (Vt) further comprises:
when the gene product of the gene is a therapeutic target of a known antagonist, and the known antagonist is a drug for a known disease, the third positive-negative correlation coefficient Vt of the gene is determined as Vt1;
when the gene product of the gene is the therapeutic target of the known antagonist, and the known antagonist is a clinical trial drug, the third positive-negative correlation coefficient Vt of the gene is determined as Vt2;
when the gene product of the gene is the therapeutic target of the known antagonist, and the known antagonist is not a clinical trial drug, the third positive-negative correlation coefficient Vt of the gene is determined as Vt3;
when the gene product of the gene is not a therapeutic target of an antagonist or not an agonist of a specific disease, the third positive-negative correlation coefficient Vt of the gene is determined as 0;
when the gene product of the gene is the therapeutic target of the known agonist, and the known agonist is not a clinical trial drug, the third positive-negative correlation coefficient Vt of the gene is determined as Vt4;
when the gene product of the gene is the therapeutic target of the known agonist, and the known agonist is a clinical trial drug, the third positive-negative correlation coefficient Vt of the gene is determined as Vt5;
when the gene product of the gene is the therapeutic target of the known agonist, and the known agonist is a drug for a known disease, the third positive-negative correlation coefficient Vt of the gene is determined as Vt6;
wherein the coefficient relationship is Vt1>Vt2>Vt3>0>Vt4>Vt5>Vt6.

5. The method for constructing a digital disease module as claimed in claim 1, wherein the step of determining the results of text mining for functions/activities of the gene product of the gene corresponding to the disease as the fourth positive-negative correlation coefficient (Vr) further comprises:
when there is literature describing that functions/activities of the gene product are positively correlated with the appearance of the disease or the functions/activities of the gene product are not beneficial to the treatment of the disease, the fourth positive-negative correlation coefficient Vr of the gene is determined as Vr1;
when there is no literature describing that the functions/activities of the gene product are positively correlated with the appearance of the disease, the fourth positive-negative correlation coefficient Vr of the gene is defined as 0;
when there is literature describing that functions/activities of the gene product are negatively correlated with the appearance of the disease or the functions/activities of the gene product are beneficial to the treatment of the disease, the fourth positive-negative correlation coefficient Vr of the gene is determined as Vr2;
wherein the coefficient relationship is Vr1>0>Vr2.

6. The method for constructing a digital disease module as claimed in claim 1, wherein the step of determining whether the gene is the upstream gene of the signaling transduction pathway and the relation with the disease as the fifth positive-negative correlation coefficient (Vu) further comprises:
determining that the gene belongs to the upstream gene when the gene product is an extracellular ligand, a cell surface receptor or a transcription factor;
adding the first, second, third, and fourth positive-negative correlation coefficients into a second sum of coefficients;
when the second sum of the coefficients is positive and the gene belongs to the upstream gene, the fifth positive-negative correlation coefficient Vu of the gene is determined as Vu1;
when the second sum of the coefficients is 0 and the gene does not belong to the upstream gene, the fifth positive-negative correlation coefficient Vu of the gene is determined as 0; and
when the second sum of the coefficients is negative and the gene belongs to the upstream gene, the fifth positive-negative correlation coefficient Vu of the gene is determined as Vu2;
wherein the coefficient relationship is Vu1>0>Vu2.

7. The method for constructing a digital disease module as claimed in claim 1, wherein the maximum value of the first, second, third, fourth, and fifth positive-negative correlation coefficients meet the following conditions:
(Vm1+Vt+Vr1)>Ve1;
(Ve1+Vt1+Vr1)>Vm1;
(Ve1+Vm1+Vr1)>Vt1;
(Ve+Vm1+Vt1)>Vr1; and
(Ve1+Vm1+Vt1+Vr1)>Vu1,
wherein when a first average of a first expression level statistical value of a gene/protein product of the gene divided by a second average of a second expression level statistical value of the gene/protein product is greater than or equal to 2, and a first statistical difference between the first expression level statistical value and the second expression level statistical value is significant, the first positive-negative correlation coefficient Ve of the gene is determined as Ve1;
wherein when the gene has more than two appearances of SNP that are negatively correlated with an appearance of the disease, a third average of statistical values of appearance rate of SNP divided by a fourth average of the statistical values of appearance rate of no SNP is greater than 1, and a second statistical difference between the statistical values of appearance rate of SNP and the statistical values of appearance rate of no SNP is significant, the second positive-negative correlation coefficient Vm of the gene is determined as Vm1;
wherein when the gene product of the gene is a therapeutic target of a known antagonist, and the known antagonist is a drug for a known disease, the third positive-negative correlation coefficient Vt of the gene is determined as Vt1;
wherein when there is literature describing that functions/activities of the gene product are positively correlated with the appearance of the disease or the functions/activities of the gene product are not beneficial to the treatment of the disease, the fourth positive-negative correlation coefficient Vr of the gene is determined as Vr1; and
when a second sum of the coefficients is positive and the gene belongs to the upstream gene, the fifth positive-negative correlation coefficient Vu of the gene is determined as Vu1, wherein the second sum of the coefficients is the sum of the first, second, third, and fourth positive-negative correlation coefficients.

8. The method for constructing a digital disease module as claimed in claim 1, wherein the minimum value of the first, second, third, fourth, and fifth positive-negative correlation coefficients meet the following conditions:
Ve4>(Vm4+Vt6+Vr2);
Vm4>(Ve4+Vt6+Vr2);
Vt6>(Ve4+Vm4+Vr2);
Vr2>(Ve4+Vm4+Vt6); and
Vu2>(Ve4+Vm4+Vt6+Vr2),
wherein when a first average of a first expression level statistical value divided by a second average of the second expression level statistical value is less than or equal to 0.5, and a first statistical difference is significant, the first positive-negative correlation coefficient Ve of the gene is determined as Ve4;
wherein when the gene has more than two appearances of SNP that are positively correlated with the appearance of the disease, a third average of the statistical values of appearance rate of SNP divided by a fourth average of the statistical values of appearance rate of no SNP is greater than 1, and a second statistical difference is significant, the second positive-negative correlation coefficient Vm of the gene is determined as Vm4;
wherein when the gene product of the gene is the therapeutic target of the known agonist, and the known agonist is a drug for a known disease, the third positive-negative correlation coefficient Vt of the gene is determined as Vt6;
wherein when there is literature describing that functions/activities of the gene product are negatively correlated with the appearance of the disease or the functions/activities of the gene product are beneficial to the treatment of the disease, the fourth positive-negative correlation coefficient Vr of the gene is determined as Vr2; and
wherein when a second sum of the coefficients is negative and the gene belongs to the upstream gene, the fifth positive-negative correlation coefficient Vu of the gene is determined as Vu2, wherein the second sum of the coefficients is the sum of the first, second, third, and fourth positive-negative correlation coefficients.

9. The method for constructing a digital disease module as claimed in claim 1, wherein the digital disease module is a three-dimensional model.

10. A device for constructing a digital disease module, comprising:
at least one processor; and
at least one computer storage media for storing at least one computer-readable instruction, wherein the processor is configured to drive the computer storage media to execute the following:
determining the relationship between changes of gene/protein expression level of a gene and a disease as a first positive-negative correlation coefficient (Ve);
determining the relationship between an appearance of single nucleotide polymorphisms (Appearance of SNP) of the gene and the disease as a second positive-negative correlation coefficient (Vm);
determining a gene product of the gene which is a target for disease suppression as a third positive-negative correlation coefficient (Vt);
determining results of text mining for functions/activities of the gene product of the gene corresponding to the disease as a fourth positive-negative correlation coefficient (Vr);
determining whether the gene is the upstream gene of the signaling transduction pathway and the relation with the disease as a fifth positive-negative correlation coefficient (Vu);
adding any three or more positive-negative correlation coefficients into a first sum of coefficients; and
constructing a digital disease module based on the first sum of coefficients to present disease genomic information.

11. The device for constructing a digital disease module as claimed in claim 10, wherein the step of determining the relationship between the changes of the gene/protein expression level of the gene and the disease as the first positive-negative correlation coefficient (Ve) performed by the processor further comprises:
determining a first expression level statistical value of a gene/protein product of the gene when the disease occurs;
determining a second expression level statistical value of the gene/protein product when the disease does not occur;
wherein when a first average of the first expression level statistical value divided by a second average of the second expression level statistical value is greater than or equal to 2, and a first statistical difference between the first expression level statistical value and the second expression level statistical value is significant, the first positive-negative correlation coefficient Ve of the gene is determined as Ve1, wherein the first statistical difference that is significant means that an independent two-sample T test <0.05 of the first expression level statistical value and the second expression level statistical value;
when the first average of the first expression level statistical value divided by the second average of the second expression level statistical value is less than 2 and greater than 1, and the first statistical difference is significant, the first positive-negative correlation coefficient Ve of the gene is determined as Ve2;
when the first statistical difference is not significant, the first positive-negative correlation coefficient Ve of the gene is determined as 0, wherein the first statistical difference that is not significant means that the independent two-sample T test ≥0.05;
when the first average of the first expression level statistical value divided by the second average of the second expression level statistical value is less than 1 and greater than 0.5, and the first statistical difference is significant, the first positive-negative correlation coefficient Ve of the gene is determined as Ve3; and
when the first average of the first expression level statistical value divided by the second average of the second expression level statistical value is less than or equal to 0.5, and the first statistical difference is significant, the first positive-negative correlation coefficient Ve of the gene is determined as Ve4;
wherein the coefficient relationship is Ve1>Ve2>0>Ve3>Ve4.

12. The device for constructing a digital disease module as claimed in claim 10, wherein the step of determining the relationship between the appearance of single nucleotide polymorphisms (Appearance of SNP) of the gene and the disease as the second positive-negative correlation coefficient (Vm) performed by the processor further comprises:
determining statistical values of appearance rate of SNP of a gene sequence of as c1˜cx;
determining statistical values of appearance rate of no SNP as d1˜dy;
wherein when the gene has more than two appearances of SNP that are negatively correlated with an appearance of the disease, a third average of the statistical values of appearance rate of SNP divided by a fourth average of the statistical value of appearance rate of no SNP is greater than 1, and a second statistical difference between the statistical values of appearance rate of SNP and the statistical values of appearance rate of no SNP is significant, the second positive-negative correlation coefficient Vm of the gene is determined as Vm1, wherein the second statistical difference that is significant means an independent two-sample T test (c1: cx, d1: dy)<0.05 of the statistical values of appearance rate of SNP and the statistical value of appearance rate of no SNP;
when the gene has more than one appearance of SNP that is negatively correlated with the appearance of the disease, the third average divided by the fourth average is greater than 1, and the second statistical difference is significant, the second positive-negative correlation coefficient Vm of the gene is determined as Vm2;
when the second statistical difference is not significant, the second positive-negative correlation coefficient Vm of the gene is determined as 0, wherein the second statistical difference that is not significant means that the independent two-sample T test ≥0.05;
when the gene has more than one appearance of SNP that is positively correlated with the appearance of the disease, the third average divided by the fourth average is greater than 1, and the second statistical difference is significant, the second positive-negative correlation coefficient Vm of the gene is determined as Vm3;
when the gene has more than two appearances of SNP that are positively correlated with the appearance of the disease, the third average divided by the fourth average is greater than 1, and the second statistical difference is significant, the second positive-negative correlation coefficient Vm of the gene is determined as Vm4;
wherein the coefficient relationship is Vm1>Vm2>0>Vm3>Vm4.

13. The device for constructing a digital disease module as claimed in claim 10, wherein the step of determining the gene product of the gene which is the target for disease suppression as the third positive-negative correlation coefficient (Vt) performed by the processor further comprises:
when the gene product of the gene is a therapeutic target of a known antagonist, and the known antagonist is a drug for a known disease, the third positive-negative correlation coefficient Vt of the gene is determined as Vt1;
when the gene product of the gene is the therapeutic target of the known antagonist, and the known antagonist is a clinical trial drug, the third positive-negative correlation coefficient Vt of the gene is determined as Vt2;
when the gene product of the gene is the therapeutic target of the known antagonist, and the known antagonist is not a clinical trial drug, the third positive-negative correlation coefficient Vt of the gene is determined as Vt3;
when the gene product of the gene is not a therapeutic target of an antagonist or not an agonist of a specific disease, the third positive-negative correlation coefficient Vt of the gene is determined as 0;
when the gene product of the gene is the therapeutic target of the known agonist, and the known agonist is not a clinical trial drug, the third positive-negative correlation coefficient Vt of the gene is determined as Vt4;
when the gene product of the gene is the therapeutic target of the known agonist, and the known agonist is a clinical trial drug, the third positive-negative correlation coefficient Vt of the gene is determined as Vt5;
when the gene product of the gene is the therapeutic target of the known agonist, and the known agonist is a drug for a known disease, the third positive-negative correlation coefficient Vt of the gene is determined as Vt6;
wherein the coefficient relationship is Vt1>Vt2>Vt3>0>Vt4>Vt5>Vt6.

14. The device for constructing a digital disease module as claimed in claim 10, wherein the step of determining the results of text mining for functions/activities of the gene product of the gene corresponding to the disease as the fourth positive-negative correlation coefficient (Vr) performed by the processor further comprises:
when there is literature describing that functions/activities of the gene product are positively correlated with the appearance of the disease or the functions/activities of the gene product are not beneficial to the treatment of the disease, the fourth positive-negative correlation coefficient Vr of the gene is determined as Vr1;
when there is no literature describing that the functions/activities of the gene product are positively correlated with the appearance of the disease, the fourth positive-negative correlation coefficient Vr of the gene is defined as 0;
when there is literature describing that functions/activities of the gene product are negatively correlated with the appearance of the disease or the functions/activities of the gene product are beneficial to the treatment of the disease, the fourth positive-negative correlation coefficient Vr of the gene is determined as Vr2;
wherein the coefficient relationship is Vr1>0>Vr2.

15. The device for constructing a digital disease module as claimed in claim 10, wherein the step of determining whether the gene is the upstream gene of the signaling transduction pathway and the relation with the disease as the fifth positive-negative correlation coefficient (Vu) performed by the processor further comprises:
determining that the gene belongs to the upstream gene when the gene product is an extracellular ligand, a cell surface receptor or a transcription factor;
adding the first, second, third, and fourth positive-negative correlation coefficients into a second sum of coefficients;
when the second sum of the coefficients is positive and the gene belongs to the upstream gene, the fifth positive-negative correlation coefficient Vu of the gene is determined as Vu1;
when the second sum of the coefficients is 0 and the gene does not belong to the upstream gene, the fifth positive-negative correlation coefficient Vu of the gene is determined as 0; and
when the second sum of the coefficients is negative and the gene belongs to the upstream gene, the fifth positive-negative correlation coefficient Vu of the gene is determined as Vu2;
wherein the coefficient relationship is Vu1>0>Vu2.

16. The device for constructing a digital disease module as claimed in claim 10, wherein the maximum value of the first, second, third, fourth, and fifth positive-negative correlation coefficients meet the following conditions:
(Vm1+Vt1+Vr1)>Ve1;
(Ve1+Vt1+Vr1)>Vm1;
(Ve1+Vm1+Vr1)>Vt1;
(Ve1+Vm1+Vt1)>Vr1; and
(Ve1+Vm1+Vt1+Vr1)>Vu1,
wherein when a first average of a first expression level statistical value of a gene/protein product of the gene divided by a second average of a second expression level statistical value of the gene/protein product is greater than or equal to 2, and a first statistical difference between the first expression level statistical value and the second expression level statistical value is significant, the first positive-negative correlation coefficient Ve of the gene is determined as Ve1;
wherein when the gene has more than two appearances of SNP that are negatively correlated with an appearance of the disease, a third average of statistical values of appearance rate of SNP divided by a fourth average of the statistical values of appearance rate of no SNP is greater than 1, and a second statistical difference between the statistical values of appearance rate of SNP and the statistical values of appearance rate of no SNP is significant, the second positive-negative correlation coefficient Vm of the gene is determined as Vm1;
wherein when the gene product of the gene is a therapeutic target of a known antagonist, and the known antagonist is a drug for a known disease, the third positive-negative correlation coefficient Vt of the gene is determined as Vt1;
wherein when there is literature describing that functions/activities of the gene product are positively correlated with the appearance of the disease or the functions/activities of the gene product are not beneficial to the treatment of the disease, the fourth positive-negative correlation coefficient Vr of the gene is determined as Vr1; and
when a second sum of the coefficients is positive and the gene belongs to the upstream gene, the fifth positive-negative correlation coefficient Vu of the gene is determined as Vu1, wherein the second sum of the coefficients is the sum of the first, second, third, and fourth positive-negative correlation coefficients.

17. The device for constructing a digital disease module as claimed in claim 10, wherein the minimum value of the first, second, third, fourth, and fifth positive-negative correlation coefficients meet the following conditions:
Ve4>(Vm4+Vt6+Vr2);
Vm4>(Ve4+Vt6+Vr2);
Vt6>(Ve4+Vm4+Vr2);
Vr2>(Ve4+Vm4+Vt6); and
Vu2>(Ve4+Vm4+Vt6+Vr2),
wherein when a first average of a first expression level statistical value divided by a second average of the second expression level statistical value is less than or equal to 0.5, and a first statistical difference is significant, the first positive-negative correlation coefficient Ve of the gene is determined as Ve4;
wherein when the gene has more than two appearances of SNP that are positively correlated with the appearance of the disease, a third average of the statistical values of appearance rate of SNP divided by a fourth average of the statistical values of appearance rate of no SNP is greater than 1, and a second statistical difference is significant, the second positive-negative correlation coefficient Vm of the gene is determined as Vm4;
wherein when the gene product of the gene is the therapeutic target of the known agonist, and the known agonist is a drug for a known disease, the third positive-negative correlation coefficient Vt of the gene is determined as Vt6;
wherein when there is literature describing that functions/activities of the gene product are negatively correlated with the appearance of the disease or the functions/activities of the gene product are beneficial to the treatment of the disease, the fourth positive-negative correlation coefficient Vr of the gene is determined as Vr2; and
wherein when a second sum of the coefficients is negative and the gene belongs to the upstream gene, the fifth positive-negative correlation coefficient Vu of the gene is determined as Vu2, wherein the second sum of the coefficients is the sum of the first, second, third, and fourth positive-negative correlation coefficients.

18. A method for constructing a digital disease module, comprising:
determining the relationship between changes of gene/protein expression level of a gene and a disease as a first positive-negative correlation coefficient (Ve);
determining the relationship between an appearance of single nucleotide polymorphisms (Appearance of SNP) of the gene and the disease as a second positive-negative correlation coefficient (Vm);
determining a gene product of the gene which is a target for disease suppression as a third positive-negative correlation coefficient (Vt);
determining results of text mining for functions/activities of the gene product of the gene corresponding to the disease as a fourth positive-negative correlation coefficient (Vr);
determining whether the gene is the upstream gene of the signaling transduction pathway and the relation with the disease as a fifth positive-negative correlation coefficient (Vu);
adding any two or more positive-negative correlation coefficients into a first sum of coefficients; and
constructing a digital disease module based on the first sum of coefficients to present disease genomic information.

19. A method for constructing a digital disease module, comprising:
determining the relationship between changes of gene/protein expression level of a gene and a disease as a first positive-negative correlation coefficient (Ve);
determining the relationship between an appearance of single nucleotide polymorphisms (Appearance of SNP) of the gene and the disease as a second positive-negative correlation coefficient (Vm);
determining a gene product of the gene which is a target for disease suppression as a third positive-negative correlation coefficient (Vt);
determining results of text mining for functions/activities of the gene product of the gene corresponding to the disease as a fourth positive-negative correlation coefficient (Vr);
determining whether the gene is the upstream gene of the signaling transduction pathway and the relation with the disease as a fifth positive-negative correlation coefficient (Vu);
adding any one or more positive-negative correlation coefficients into a first sum of coefficients; and
constructing a digital disease module based on the first sum of coefficients to present disease genomic information.
